(19) United States
(12) Patent Application Publication
(10) Pub. No.: US 2014/0163540 A1
(43) Pub. Date: Jun. 12, 2014

(54) APPARATUS FOR EFFECTING RENAL DENERVATION USING ULTRASOUND

(71) Applicants: Mano Iyer, Staten Island, NY (US); Jaime E. Merino, Mountain View, CA (US); Kenneth J. Maynard, East Islip, NY (US)

(72) Inventors: Mano Iyer, Staten Island, NY (US); Jaime E. Merino, Mountain View, CA (US); Kenneth J. Maynard, East Islip, NY (US)

(73) Assignee: ReCor Medical, Inc., Menlo Park, CA (US)

(21) Appl. No.: 14/000,168

(22) PCT Filed: Feb. 18, 2011
(86) PCT No.: PCT/US2011/025543

§ 371 Date: Dec. 20, 2013

(51) Int. Cl. A61N 7/02

(52) U.S. Cl. CPC A61N 7/022
USPC 606/27

(57) ABSTRACT

An ultrasound apparatus is provided for non-continuous circumferential treatment of a vessel, such to provide denervation of a renal blood vessel. The apparatus may be positioned within a vessel to deliver broad unfocused or focused ultrasonic energy at first and second lengthwise and angular positions to create a less-than-full circumferential treatment zone at each of the first and second positions. Superimposition of treatment zones defines a non-continuous circumferential treatment zone without formation of a continuous circumferential lesion.

### I. FIELD OF THE INVENTION

This application relates to apparatus for performing non-continuous circumferential denervation of the renal arteries to treat a variety of renal and cardiac related diseases, including cardio-renal syndrome, heart failure, sudden cardiac death, left ventricular hypertrophy, renal disease, renal failure, hypertension, contrast nephropathy, cirrhosis, arrhythmia and myocardial infarction, using ultrasound treatment modalities.

### II. BACKGROUND OF THE INVENTION

Methods and apparatus are known for treating a variety of renal and cardio-renal diseases, such as cardio-renal syndrome, heart failure, sudden cardiac death, left ventricular hypertrophy, renal disease, renal failure, hypertension, contrast nephropathy, cirrhosis, arrhythmia and myocardial infarction, by modulating neural fibers that contribute to renal function, and in particular, by denervating tissue containing the neural fibers that contribute to renal function. This is expected to reduce renal sympathetic nervous activity, thereby increasing removal of water and sodium from the body, and returning renin secretion to more normal levels. A number of published patent applications suggest that normalized renin secretion causes blood vessels supplying the kidneys to assume a steady state level of dilation/constriction, which provides adequate renal blood flow. See, for example, U.S. Patent Application Publication Nos. US 2003/0216792, US 2005/0288730, and US 2006/0276852 and U.S. Pat. No. 6,978,174. All of these applications and the patent are incorporated herein by reference in their entireties.

Methods and apparatus also are known for intravascularly-inducing neuromodulation or denervation of an innervated blood vessel in a patient or any target neural fibers in proximity to a blood vessel, for example, to treat any neurological disorder or other medical condition. Nerves in proximity to a blood vessel may innervate an effector organ or tissue. Intravascularly-induced neuromodulation or denervation may be utilized to treat a host of neurological disorders or other medical conditions, including, but not limited to, the aforementioned conditions including heart failure and hypertension, as well as pain and peripheral arterial occlusive disease (e.g., via pain mitigation). The methods and apparatus may be used to modulate efferent or afferent nerve signals, as well as combinations of efferent and afferent nerve signals. See, for example, U.S. Patent Application Publication No. US 2007/0129760, which is incorporated herein by reference in its entirety.

Although the methods and apparatus described in the foregoing patent publications and patent appear advantageous, one challenge of neuromodulation and/or denervation is sufficiently affecting the neural tissue from within the vessel. For example, intravascular neuromodulation should avoid increasing the risk of acute and/or late stenosis. Therefore, it would be desirable to provide methods and apparatus that further address these challenges.

For example, U.S. Patent Application Publication Nos. US 2007/0129720 and US 2010/0137860 to Demarais et al. describe methods and apparatus for denervating a vessel in a non-continuous circumferential pattern, which is described as potentially reducing a risk of acute and/or late stenosis formation within the vessel caused by treating the full circumference of the vessel at a given longitudinal position. Those publications mention that high intensity focused ultrasound could be used in apparatus therein and for the described methods, but do not provide any details regarding how such an ultrasound device could be constructed.

U.S. Pat. No. 6,913,581 to Corl describes a catheter including a distal region carrying a plurality of ultrasound transducers. Each of the plurality of transducers disclosed in that patent emits energy in around its full circumference, and accordingly that catheter could not be used to provide a non-continuous circumferential energy pattern as described in the Demarais publications.

In view of the foregoing, it would be desirable to provide an ultrasound catheter having a plurality of ultrasound transducers arranged on or within a distal region of the catheter and configured to emit ultrasonic energy in a non-continuous circumferential pattern.

It further would be desirable to provide an ultrasound catheter having a plurality of ultrasound transducers arranged on or within a distal region of the catheter, wherein adjacent transducers are configured to emit ultrasonic energy over adjacent arcs of the vessel circumference.

It also would be desirable to a method of making an ultrasound catheter configured to emit ultrasonic energy in a spiral pattern.

### III. SUMMARY OF THE INVENTION

The present invention overcomes the drawbacks of previously-known apparatus and methods for forming non-continuous denervation patterns within vessels. In accordance with the principles of the present invention, ultrasound catheters are provided in which a unitary spiral ultrasound transducer, or a plurality of ultrasound transducers, is disposed along a distal region of a catheter, and configured to emit ultrasonic energy over adjacent circumferential arcs of the vessel. In this manner, the benefits of renal neural modulation or denervation can be achieved while reducing the risk of restenosis or potential vessel wall thinning resulting from full circumferential denervation.

In accordance with one aspect of the present invention, a plurality of curved or flat ultrasound transducers are disposed on a distal region of an intravascular catheter, such that each transducer emits energy only over predetermined arc and for a predetermined length of a vessel. In alternative embodiments, the distal region of the catheter may include a series of flat or curved transducers disposed within the catheter and covered by acoustically transmissive windows, such that each transducer emits energy only over a predetermined arc and along a predetermined length of a vessel.

In other embodiments, the distal region of the catheter may include a series of circumferential transducers, with adjacent transducers covered over predetermined arcs by an acoustically absorptive material, such that each transducer emits energy only over a predetermined arc and for a predetermined length of a vessel. The absorptive material also may be disposed as a spiral stripe that extends along the distal region, such that ultrasonic energy is emitted primarily through the uncovered portion of the distal region to create a spiral longitudinally extending lesion. In further alternative embodiments, the distal region of the catheter may include a series of circumferential transducers, with adjacent transducers covered over predetermined arcs by an acoustically reflective material, such that energy emitted by each transducer is directed only over a predetermined arc and for a predetermined length of a vessel.

Finally, the ultrasound transducer may comprise a single or multiple spiral shapes that extend along a distal region of a catheter, e.g., formed by cutting or scribing a piezoelectric transducer. Alternatively, the spiral transducer may be formed by wrapping a piezoelectric film around the exterior of a catheter in a spiral configuration. Using the foregoing catheters, neural fibers disposed within a vessel may be modulated and/or ablated in a non-continuous circumferential, spiral pattern, over a selected length of the vessel, such that the lesions formed thereby are not contiguous or continuous about any complete circumference of a cross-section normal to a longitudinal axis of the vessel.

Methods of making and using the ultrasound catheters of the present invention also are provided.

### IV. BRIEF DESCRIPTION OF THE DRAWINGS

Several embodiments of the present invention will be apparent upon consideration of the following detailed description, in which:

FIG. 1 is a schematic view showing a common location of neural fibers proximate an artery.

FIG. 2 is a schematic side view of a catheter of the present invention for non-continuous circumferential treatment of a vessel.

FIG. 3 is a cross-sectional view of a first embodiment of an ultrasound transducer suitable for use in the catheter of FIG. 1.

FIG. 4 is a cross-sectional view of a second embodiment of an ultrasound transducer suitable for use in the catheter of FIG. 1.

FIGS. 5A and 5B are cross-sectional views of further alternative embodiments of ultrasound transducers suitable for use in the catheter of FIG. 1.

FIGS. 6A and 6B are perspective views showing additional embodiments employing the transducers depicted in FIG. 5A or 5B.

FIGS. 7A and 7B are, respectively, cross-sectional and partial perspective views showing an embodiment in which flat ultrasound transducers are disposed within a catheter and emit ultrasonic energy through acoustically transmissive windows.

FIG. 8 is a cross-sectional view showing an embodiment in which curved ultrasound transducers are disposed within a catheter and emit ultrasonic energy through acoustically transmissive windows.

FIG. 9 is a perspective view showing a further alternative embodiment of a catheter constructed in accordance with the principles of the present invention wherein the ultrasonic transducer comprises cylindrical transducers stacked in a side by side arrangement.

FIG. 10 is a perspective view showing a further alternative embodiment of a catheter constructed in accordance with the principles of the present invention wherein the ultrasonic transducer has a spiral configuration.

### V. DETAILED DESCRIPTION OF EMBODIMENTS OF THE INVENTION

The apparatus of the present invention comprises an ultrasound catheter having one or multiple transducers disposed on a distal region of the catheter to provide neural modulation and/or denervation of a blood vessel, such as a renal artery, for therapeutic purposes known in the art. As described in the above-mentioned published patent applications, it may be desirable to perform a circumferential treatment of a body lumen to positively affect a medical condition by applying energy to discrete zones that are non-continuous along the complete circumference of a radial cross-section generally normal to the lumen wall.

For example, in the treatment of atrial fibrillation or other arrhythmia, a circumferential treatment may be achieved by forming a continuous circumferential lesion that is continuous completely about a normal cross-section of the pulmonary vein to disrupt aberrant electrical signals. In the treatment of heart failure, a circumferential treatment may be achieved by forming a similar continuous circumferential lesion that is continuous completely about a normal cross-section of a renal artery to reduce renal sympathetic neural activity. However, continuous circumferential lesions that extend continuously about a full 360° of the circumference of a cross-section normal to the body lumen or tissue in proximity to the body lumen may increase a risk of acute and/or late stenosis formation within the vessel. The catheter of the present invention is directed to forming discrete, non-continuous lesions normal of a lumen without adversely affecting the mechanical integrity of the vessel or adversely impacting the potential for stenosis of the vessel.

In accordance with one aspect of the invention, treatments may be applied relative to nerves within or in proximity to a blood vessel that travel at least generally parallel or along a lengthwise dimension of the blood vessel. The target structures may additionally or alternatively include a rotational orientation relative to the blood vessel. The disclosed embodiments may reduce the risk of acute and/or late stenosis formation by treating neural matter along portions of multiple radial planes or cross-sections that are normal to, and spaced apart along, the lengthwise or longitudinal axis of the blood vessel.

The treatment area at each radial plane or cross-section defines a treatment zone that is not completely continuous along a normal circumference, i.e., defines a treatment zone without a continuous circumferential lesion normal to the longitudinal axis. However, superimposition of the multiple treatment zones along the multiple radial planes or normal cross-sections defines a non-continuous, overlapping circumferential treatment zone along a lengthwise or longitudinal segment of the blood vessel. In some embodiments, this overlapping treatment zone may provide a non-continuous, but substantially fully circumferential treatment without formation of a continuous circumferential lesion normal to the vessel. In other embodiments, the overlapping treatment zone may provide a non-continuous, partial circumferential treatment.

In this manner, a non-continuous circumferential treatment is performed over a lengthwise segment of the blood vessel, as compared to a continuous circumferential treatment at a single normal cross-section or radial plane. Target structures substantially traveling along the lengthwise dimension of the blood vessel are thus circumferentially affected in a non-continuous fashion without formation of the continuous circumferential lesion along any normal cross-section or radial plane of the blood vessel. A non-continuous circumferential treatment thus may comprise a treatment conducted at multiple positions about the lengthwise dimension of a vessel, wherein the treatment zone at any one lengthwise position does not comprise a continuous circumferential lesion completely about a radial plane or normal cross-section. However, a superimposition of the treatment zones at all or some of the lengthwise positions may define an overlapping circumferential treatment zone.

Treatment may be achieved via either direct alteration of the target structures (e.g., target neural structures) or at least in part via alteration of the vascular or other structures that support the target structures or surrounding tissue, such as arteries, arterioles, capillaries, veins or venules. In some embodiments, the treatment may be achieved via direct application of energy to the target or support structures, e.g., ultrasound energy that causes resistive heating in the target or supporting structures.

In some embodiments, apparatus for real-time monitoring of the treatment and its effects on the target or support structures, and/or in non-target tissue, may be provided. Likewise, real-time monitoring of the energy delivered by the catheter may be provided. For example, power or total energy delivered, or the temperature or other characteristic of the target or the non-target tissue may be monitored. Feedback, such as sensed temperature, along target or non-target tissue or along the apparatus, optionally may be used to control and monitor delivery of the thermal energy.

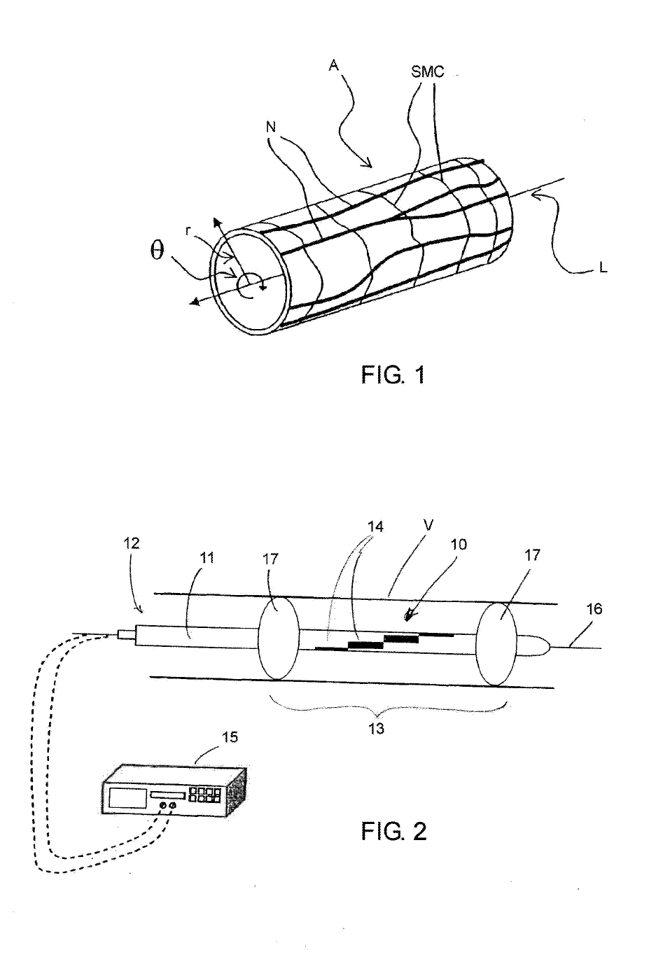

Referring to FIG. 1, a common anatomical arrangement of neural structures relative to vascular structures, such as arteries, is described. Neural fibers N generally may extend longitudinally along the lengthwise dimension L of an artery A about a relatively small range of positions along the radial dimension r, often within the adventitia of the artery. The artery A has smooth muscle cells SMC that surround the arterial circumference and generally spiral around the angular dimension 0 of the artery, also within a relatively small range of positions along the radial dimension r. The smooth muscle cells of the artery accordingly have a lengthwise or longer dimension generally extending transverse (i.e., non-parallel) to the lengthwise dimension of the blood vessel. Misalignment of the lengthwise dimensions of the neural fibers and the smooth muscle cells is referred to herein as “cellular misalignment.”

The cellular misalignment of the nerves N and the smooth muscle cells SMC may be exploited to selectively affect the nerve cells with reduced effect on the smooth muscle cells. More specifically, a non-continuous circumferential treatment may be achieved by superimposing treatments undertaken along multiple radial or cross-sectional planes of the artery A that are separated along the lengthwise dimension L of the artery, rather than performing a continuous circumferential treatment along a single radial plane or cross-section of the artery. In this manner, due to the cellular misalignment, the lengthwise-oriented neural fibers may experience a full, non-continuous circumferential treatment, while the angularly-oriented smooth muscle cells may experience only a partial circumferential treatment. Monitoring elements optionally may be utilized to assess an extent of treatment induced in the nerves and/or in the smooth muscle cells, as well as to adjust treatment parameters to achieve a desired effect.

Referring now to FIG. 2, a catheter constructed in accordance with the principles of the present invention is now described. Apparatus 10 comprises catheter 11 having proximal region 12, distal region 13 and a plurality of ultrasound transducers 14 disposed in distal region 13. Ultrasound transducers 14 are coupled via connectors to power supply and controller 15, such as are known in the art, which supplies the energy to power transducers 14. Catheter 11 includes a guide wire lumen that accepts guide wire 16, thereby enabling catheter to negotiate the directional change encountered where, e.g., the ostia of the renal arteries join the descending abdominal aorta. Catheter 11 may include optional expandable members 17, illustratively balloons or mechanically expandable cages, which may be reversibly deployed to center catheter 11 within vessel V. Catheter 11 also may include temperature sensors disposed in distal region 13, such as are known in the art, that are configured to measure the temperature of the vessel and surrounding tissue, to monitor heating caused by disposition of ultrasonic energy.

In accordance with one aspect of the present invention, transducers 14 are disposed within distal region 13 such that each transducer extends for a predetermined length, e.g., 10 mm, and emits energy only over a predetermined arc, e.g., illustratively, from about 30° to about 90° of the circumference of the vessel. In a preferred embodiment, transducers 14 spiral around the exterior of catheter 11, as depicted in FIG. 2, and are arranged to cover adjacent arcs of the vessel circumference. Preferably, the arcs of the plurality of transducers emit energy that, in the aggregate, covers the entire circumference of the vessel over the length of distal region 13. When catheter is disposed within vessel V, as depicted in FIG. 2, and energized, it creates a pattern of lesions within the vessel wall and surrounding neural fibers corresponding to the locations of the transducers that is circumferentially non-continuous at any given position along the length of the artery. In the aggregate, however, the pattern of lesions covers the entire circumference of the vessel of a length corresponding to the length of distal region 13.

Alternatively, transducers 14 need not be configured to emit energy directed at adjacent arcs of the circumference, but could instead be staggered around the exterior of catheter 11 in distal region 13. However, so as to ensure that there is a complete denervation of the neural fibers in the vessel, the aggregate of the arcs treated by transducers 14 preferably covers the entire circumference. As a further alternative, transducers 14 may be disposed such that at each axial location, multiple individually energizable transducers are disposed on the exterior of the catheter. In this case, the power supply and controller 15 may be arranged so that, at a given axial location within distal region 13, every other (or second, third, etc.) transducer may be energized and the transducers activated for more proximal or distal axial segments may be staggered, again creating a pattern of lesions that encompasses the entire circumference of the vessel.

In preferred embodiments, the ultrasonic transducers are operated in a range of from 1 to 20 MHz, and more preferably at about 9 MHz, and deliver from 5 to 80 Watts of power to the tissue being remodeled, and more preferably 5 to 50 Watts. Energy delivery preferably occurs over a period of from 30 seconds to 5 minutes, more preferably from about 1 to 3 minutes, such that the temperature achieved in the target nerve structures is sufficient to cause remodeling of the collagen, but without causing substantial necrosis of the adjacent tissue. In one preferred embodiment, the catheter of the present invention is configured to simultaneously deliver energy at up to 6 longitudinal locations along a renal artery to provide a satisfactory conduction block in the adjacent neural tissue.

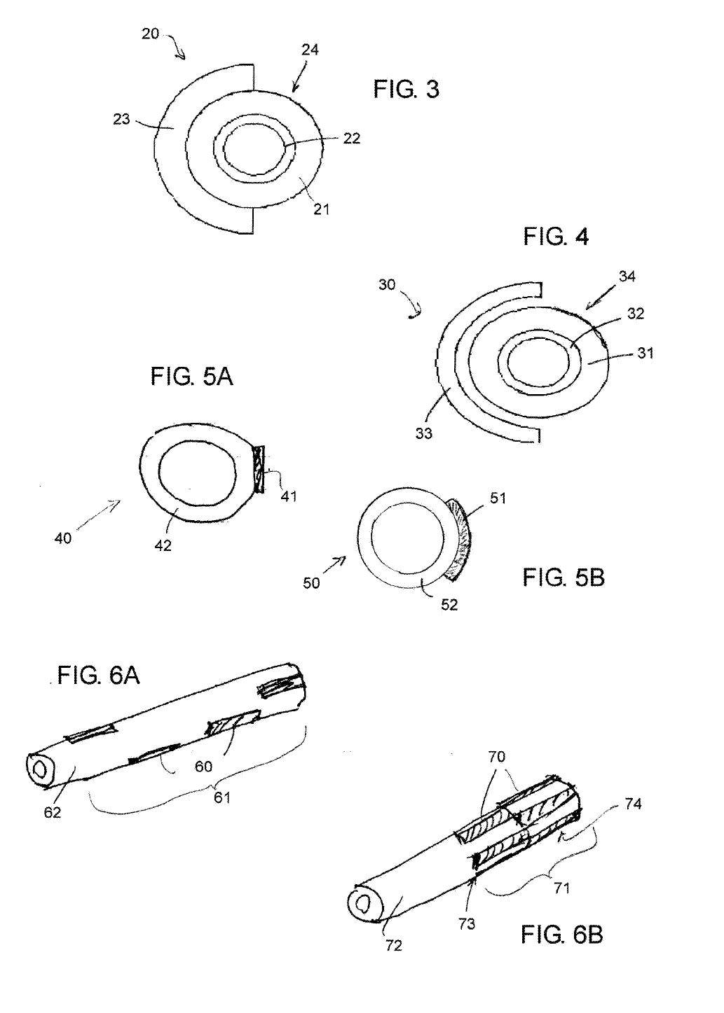

Referring now to FIG. 3, transducer 20, which corresponds to one of transducers 14 of FIG. 2 and is suitable for use in making apparatus 10 of the present invention, is described. Transducer 20 may be constructed of layer 21 of piezoelectric material, as described, for example, in commonly assigned U.S. Pat. No. 7,837,676 to Sinelnikov, the entirety of which is incorporate herein by reference. Layer 21 extends around the circumference of catheter 22, and in addition may include a rigid backing layer and other structures conventionally used in making catheter-based transducers, which are omitted for clarity. In accordance with one aspect of the present invention, however, layer 21 further includes layer 23 of absorptive material disposed over a predetermined arc of its outer surface, e.g., from 180° to about 330° (illustratively 180° in FIG. 3). Layer 23 preferably comprises a biocompatible rubber or similar material, and is configured to absorb the ultrasonic energy emitted by the underlying portion of transducer. Accordingly, when energized, transducer 20 emits energy primarily only through its exposed face 24. As will be understood, a plurality of transducers 20 preferably are disposed on adjacent axial segments of the distal region of the catheter, such that the ultrasonic energy emitted by the transducers results in non-continuous circumferential neural modulation or denervation of the vessel at a particular axial location, but in the aggregate covers the entire circumference of the vessel over the length of the distal region.

Alternatively, layer 23 of absorptive material may be disposed as a spiral or longitudinal stripe over a substantially cylindrical transducer that extends longitudinally for the length of the distal region. In this case, ultrasonic energy is emitted from the transducer primarily through the uncovered spiral or longitudinal portions of the distal region when energized. In the case where the absorptive material is disposed in a spiral stripe, the emitted ultrasonic energy will produce a substantially longitudinally extending spiral lesion in the vessel. Alternative transducer designs for generating a spiral lesion in the vessel wall are described below with respect to the embodiments of FIG. 10. As a further alternative, layer 23 of absorptive material may be arranged as circumferentially spaced apart longitudinally stripes, or a plurality of circular, rectangular, elliptical, etc., holes in the layer of absorptive material, so that the uncovered portions of the transducer create a similarly-shaped lesion pattern in the vessel when energized. As should be appreciated, alternative patterns of layer 23 of absorptive material may be disposed on the transducer to obtain a desired size and shape of the resulting lesion.

With respect to FIG. 4, transducer 30, again corresponding to single one of transducers 14 of FIG. 2 is described. Transducer 30 may be similar in construction to the transducer 20 as described above, and includes layer 31 of ultrasound emitting material that extends around the circumference of the catheter 32. Transducer 30 differs from the embodiment of FIG. 3, however, in that it includes layer 33 of reflective material disposed over a predetermined arc of its outer surface, e.g., from 180° to about 330° (illustratively 180° in FIG. 4). Layer 33 preferably comprises a material that creates an acoustic mismatch with layer 31 that reflects the ultrasonic energy emitted by the underlying portion of transducer. For example, layer 33 may comprise an air-filled reflective layer, as described in the above-incorporated U.S. Pat. No. 7,837,676. Accordingly, when energized, transducer 30 emits energy primarily only through its exposed face 34, while energy emitted by the remainder of the ultrasound emitting layer 31 is reflected by layer 33. As will be understood, a plurality of transducers 30 may be disposed on adjacent axial segments of the distal region of the catheter, such that the ultrasonic energy emitted by the transducers results in non-continuous circumferential neural modulation or denervation of the vessel at a particular axial location, but in the aggregate covers the entire circumference of the vessel over the length of the distal region.

FIGS. 5A and 5B depict further alternative embodiments of transducers suitable for use in constructing the catheter of FIG. 2. Transducer 40 of FIG. 5A preferably comprises a flat layer 41 of piezoelectric material affixed to the exterior of catheter 42; the wires for connecting transducer 40 to power supply and controller 15 (see FIG. 1) have been omitted for clarity. Layer 41 may be formed on a flat, suitably rigid substrate, and then fastened to the exterior of catheter 42 using a biocompatible adhesive or epoxy. As will be understood, transducer 40, when energized, will emit ultrasonic energy substantially orthogonal to the face of layer 41, resulting in modulation or ablation of neural fibers in the path of the emitted energy. Layer 41 may subtend a predetermined arc of the exterior of catheter 42, illustratively about 45° in FIG. 5A. In accordance with one aspect of the present invention, a number of transducers may be affixed to the exterior of catheter 42 along the length of the distal region, as depicted in FIG. 2, such that the ultrasonic energy emitted by the transducers results in non-continuous circumferential neural modulation or denervation of the vessel at a particular axial location, but in the aggregate covers the entire circumference of the vessel over the length of the distal region.

Transducer 50 of FIG. 5B preferably comprises a curved layer 51 of piezoelectric material affixed to the exterior of catheter 52; the wires for connecting transducer 50 to power supply and the controller again being omitted for clarity. Layer 51 may be formed on a suitable rigid tubular substrate, and then cut longitudinally to form separate segments that each span a predetermined arc, e.g., from about 30° to about 90°. Layer 51 then may be fastened to the exterior of catheter 52 using a biocompatible adhesive or epoxy. Transducer 50, when energized, emits ultrasonic energy substantially orthogonal to the face of layer 51, resulting in modulation or ablation of neural fibers in the path of the emitted energy. As for preceding embodiments, a plurality of transducers 50 may be affixed to the exterior of catheter 52 along the length of the distal region, such that the ultrasonic energy emitted by the transducers results in non-continuous circumferential neural modulation or denervation of the vessel at a particular axial location. In the aggregate, however, the energy emitted by the plurality of transducers covers the entire circumference of the vessel over the length of the distal region.

Referring now to FIGS. 6A and 6B, various arrangements of the transducers described in FIGS. 5A and 5B are described. In FIG. 6A, a plurality of transducers 60 are disposed along the length of distal region 61 of catheter 62 in a circumferentially staggered configuration, similar to that depicted in FIG. 2. In FIG. 6B, a plurality of transducers 70 are arranged in distal region 71 of catheter 72 in two rows, 73 and 74, where in each row the transducers alternate with blank spaces around the circumference of the catheter. The arrangement of FIG. 6B may be particularly advantageous for use in short arteries, such as the renal arteries. Alternatively, the power supply and controller may be configured to individually energize transducers 70 within rows 73 and 74, thereby enabling the physician to create patient specific complex lesion patterns in the surrounding neural fibers.

With respect to the embodiments of FIGS. 6A and 6B, treatment may first be undertaken at a first angular orientation with in a target vessel, and additional angular and/or lengthwise positions relative to the vessel wall may be achieved by rotation and lengthwise repositioning of the distal region of the catheter. This may be repeated at as many lengthwise and/or angular positions as desired by the healthcare provider, such that the treatment at each lengthwise position preferably do not form a continuous circumferential lesion normal to the vessel wall. Superimposition of the treatments at multiple such lengthwise positions preferably forms a non-continuous, partially or fully circumferential lesion, as described previously.

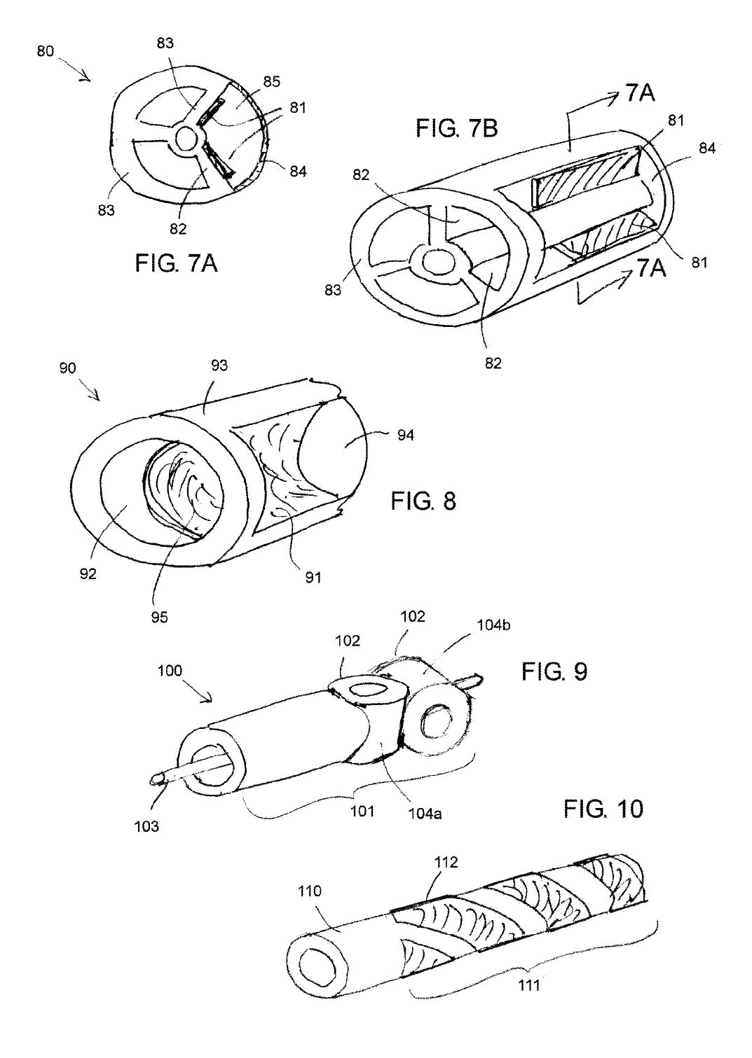

Referring now to FIGS. 7A and 7B, a further alternative embodiment of an ultrasound transducer suitable for use making the catheter of FIG. 2 is described. FIG. 7A provides a cross-sectional view of transducer 80, while FIG. 7B provides a perspective view, partly in section, of the transducer as it would appear along a single axial section of the distal region of the catheter. Transducer 80 preferably comprises one or more flat layers 81 of piezoelectric material affixed to radially directed ribs 82 formed within catheter 83; the wires for connecting transducer 80 to the power supply and controller being omitted for clarity. Layers 81 may be formed on a flat, suitably rigid substrate, and then fastened to ribs 82 of catheter 83 using a biocompatible adhesive or epoxy. Catheter 83 may include acoustically transmissive window 84 disposed over the layers 81, and space 85 enclosed by window 84 may be filled with an acoustically transmissive fluid, such as water. When energized, transducer 80 emits ultrasonic energy that passes through window 84 and impinges upon, and modulates or ablates neural fibers in the vessel wall and supporting structures.

The number, dimensions and placement of ribs 82, layers 81, and window 84 may be selected to extend over a predetermined arc of the exterior of catheter 83, illustratively about 120° in FIG. 7A. As for preceding embodiments of the present invention described above, a number of angularly and axially offset transducers 80 may be formed in the distal region of the catheter, e.g., as depicted in FIG. 2, such that the ultrasonic energy emitted by the transducers, in either an intersecting focused pattern of ultrasound energy or a broad unfocused pattern of ultrasound energy, results in non-continuous circumferential neural modulation or denervation of the vessel at a particular axial location, but in the aggregate covers the entire circumference of the vessel over the length of the distal region.

With respect to FIG. 8, another alternative embodiment of an ultrasound transducer suitable for use making the catheter of FIG. 2 is described. FIG. 8 provides a perspective view similar to that of FIG. 7B, partly in section, of transducer 90 as it would appear along a single axial section of the distal region of the catheter. Transducer 90 preferably comprises a curved layer 91 of piezoelectric material affixed to the interior wall 92 of catheter 93; the wires for connecting transducer 90 to the power supply and controller again being omitted for clarity. Layer 91 may be formed on a flat flexible substrate and then fastened to interior wall 92 of catheter 93 using an adhesive or epoxy. Alternatively, layer 91 may be formed on a curved rigid substrate that is dimensioned to mate with the curvature of interior wall 92, and then fastened into position. As a further alternative, layer 91 may be fastened to an internal support structure formed within the catheter. Catheter 93 preferably includes acoustically transmissive window 94 disposed over the layers 81, and space 95 enclosed by window 94 may be filled with an acoustically transmissive fluid, such as water. When energized, transducer 90 emits ultrasonic energy that passes through window 94 and impinges upon, and modulates or ablates, neural fibers in the vessel wall and supporting structures.

The dimensions and placement of layers 91 and windows 94 may be selected to extend over a predetermined arc of the exterior of catheter 93, illustratively about 120° in FIG. 8. As for preceding embodiments, a number of angularly and axially offset transducers 90 may be formed in the distal region of the catheter 93, e.g., as depicted in FIG. 2, such that the ultrasonic energy emitted by the transducers, in either an intersecting focused pattern of ultrasound energy or a broad unfocused pattern of ultrasound energy, results in non-continuous circumferential neural modulation or denervation of the vessel at a particular axial location, but in the aggregate covers the entire circumference of the vessel over the length of the distal region.

Referring now to FIG. 9, yet another alternative embodiment of an ultrasound catheter suitable for forming a non-continuous circumferential lesion in a vessel wall is described. Catheter 100, of which only distal region 101 is shown in FIG. 9, comprises a plurality of toroidal transducers 102 stacked in a side-by-side angularly offset configuration. Transducers 102 may be constructed as described above with respect to the piezoelectric component of FIGS. 3 and 4, and then bolted together on centrally disposed support rod 103 in a side-by-side, angularly offset relation. The wires for connecting transducers 102 to the power supply and controller, and other support structures, whic have been omitted from FIG. 9 to improve clarity. When catheter 100 is energized, each transducer 102 emits ultrasonic energy from its exposed circumferential area 104a, 104b, etc., which impinges upon, and modulates or ablates, neural fibers in the vessel wall and supporting structures. As for preceding embodiments, the dimensions and angular orientations of transducers 102 preferably are selected so that ultrasonic energy emitted by the transducers results in neural modulation or denervation over the entire circumference of the vessel, over the length of the distal region.

FIG. 10 illustrates another embodiment of an ultrasound catheter, constructed in accordance with the principles of the present invention. Catheter 110, of which only distal region 111 is shown in FIG. 10, comprises one or more ultrasonic transducers 112 that spiral along distal region 111 of the catheter. Transducer 112 preferably comprises a piezoelectric material, such as is used in the manufacture of previously-known ultrasound catheters. In one embodiment, transducer 112 is formed by depositing piezoelectric material on a flexible substrate, which then is wrapped around the exterior of catheter 110 and fastened in place. Alternatively, transducer 112 may be formed by depositing piezoelectric material on the exterior of a rigid tubular substrate. A longitudinally extending spiral then may be cut from the tube and removed, leaving longitudinally extending spiral transducer 112, which may then be affixed to the exterior of catheter 110 using a suitable biocompatible adhesive. As a yet further alternative, multiple spiral transducers may be formed on a single tube by cutting or scribing the piezoelectric material and then electrically isolating the spirals. In this manner, multiple individually energizable angularly offset transducers may be formed in distal region 111 and affixed to the catheter. When catheter 110 is energized, transducer(s) 112 emits ultrasonic energy that impinges upon, and modulates or ablates, neural fibers in the vessel wall and supporting structures.

The width, pitch and number of spiral transducers 112 of catheter 110 preferably are selected so that ultrasonic energy emitted by the transducers results in neural modulation or denervation over the entire circumference of the vessel, over the length of the distal region. In particular, the used of multiple spirals may facilitate formation of multiple, non-continuous treatment zones along one or more normal radial planes of the vessel. Continuous or non-continuous oblique treatments also may be achieved, while non-continuous normal circumferential treatments may be achieved by superimposing treatment at multiple locations (either discrete or continuous) along a lengthwise segment of the vessel.

As discussed previously, non-continuous circumferential treatment by positioning transducers at different angular orientations along multiple lengthwise locations may preferentially affect anatomical structures that substantially propagate along the lengthwise dimension of the artery. Such anatomical structures preferably are neural fibers and/or structures that support the neural fibers. Furthermore, such a non-continuous circumferential treatment may mitigate or reduce potentially undesirable effects induced in structures that propagate about the angular dimension of the artery, such as smooth muscle cells. The angular or circumferential orientation of the smooth muscle cells relative to the artery may at least partially explain why continuous circumferential lesions may increase a risk of acute or late stenosis.

With any of the catheter embodiments described herein, during delivery of the ultrasonic energy, blood within the vessel may act as a thermal sink for conductive and/or convective heat transfer by removing excess thermal energy from non-target tissue (such as the interior wall of the vessel), thereby protecting the reducing the risk of inducing hyperthermia in the vessel wall. This effect may be enhanced when blood flow is not obstructed during sonication, for example, by optional balloons 17 in the embodiment of FIG. 2. Accordingly, other expandable devices, such as expandable struts or multi-lobed balloons may be used to center the catheter within the vessel while still providing some blood flow through the vessel. Use of the patient's blood as a thermal sink also may facilitate delivery of longer or higher energy treatments with reduced risk of damage to the non-target tissue, which may enhance the efficacy of the treatment at the target tissue, for example, at target neural fibers.

In addition or as an alternative to utilizing the patient's blood as a thermal sink, a fluid may be injected, infused or otherwise delivered into the vessel to remove excess thermal energy and protect the non-target tissues. The fluid may comprise, for example, chilled or room temperature saline (e.g., saline at a temperature lower than the temperature of the vessel wall during the therapy delivery) and may be injected through the catheter or an associated guide catheter. Such fluid injection may be in the presence of blood flow within the vessel, or with flow temporarily occluded. Occlusion of flow in combination with fluid delivery may facilitate better control over the heat transfer kinetics along the non-target tissues, as well as optional injection of the fluid from a downstream location.

As described herein, a continuous circumferential lesion is a circumferential lesion that is substantially continuous in a radial plane normal to the vessel or luminal wall. Conversely, a non-continuous circumferential lesion may be non-continuous relative to a normal radial plane, but substantially continuous along an oblique plane of the vasculature that is not normal to the vessel wall. For example, an oblique circumferential treatment may be achieved within the patient's vasculature, e.g., the patient's renal artery, without formation of a continuous circumferential treatment relative to a normal radial plane of the vasculature.

Although preferred illustrative variations of the present invention are described above, it will be apparent to those skilled in the art that various changes and modifications may be made thereto without departing from the invention. For example, in the described embodiments of FIGS. 1-6, non-continuous circumferential treatment may be achieved via superimposition of treatment at two locations. In addition, treatment at more than two locations may be superimposed to achieve the circumferential treatment. It is intended in the appended claims to cover all such changes and modifications that fall within the true spirit and scope of the invention.

1-18. (canceled)

19. A system configured to selectively denervate tissue surrounding a lumen, the system comprising:
a catheter comprising an ultrasonic transducer positioned proximate a distal end of the catheter, the ultrasonic transducer being configured to selectively emit ultrasonic energy radially outwardly from an outer surface of the ultrasound transducer, the system further comprising a plurality of covering layers of absorptive material positioned radially in spaced fashion along the outer surface of the ultrasound transducer, the covering layers configured to block the emission of ultrasonic energy radially outwardly away from the ultrasonic transducer at locations where the covering layers are positioned.

20. The system of claim 19, wherein the covering layers are radially positioned in a spaced helical fashion along the outer surface of the ultrasound transducer.

21. The system of claim 19, wherein the absorptive material comprises a biocompatible rubber.

22. The system of claim 19, wherein each of the covering layers comprises a continuous helical stripe extending at least partially over the outer surface of the ultrasound transducer, wherein each covering layer is axially spaced from an adjacent covering layer.

23. The system of claim 19, wherein at least one of the covering layers comprises a plurality of openings through which ultrasound energy is permitted to pass radially outwardly from the ultrasound transducer when the ultrasound transducer is energized.

24. The system of claim 19, wherein the ultrasound transducer comprises a cylindrical shape.
